(19) United States
(12) Patent Application Publication
(10) Pub. No.: US 2009/0035331 A1
(43) Pub. Date: Feb. 5, 2009

(54) Systems and Methods for Targeted and Controlled Delivery of Agents

(75) Inventors: Eugenia Wang, Prospect, KY (US); Bruce Alphenaar, Louisville, KY (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 11/996,793

(22) PCT Filed: Jan. 29, 2007
(86) PCT No.: PCT/US2007/061243

§ 371 Date: Jan. 25, 2008

Related U.S. Application Data
(60) Provisional application No. 60/763,011, filed on Jan. 27, 2006.

(51) Int. Cl. A61K 9/00; A61K 38/02; A61K 48/00; A61K 39/395; A61K 35/76; A61K 31/7105

(52) U.S. Cl. 424/400; 514/2; 514/44; 424/130.1; 424/93.6

(57) ABSTRACT

An Agent Delivery System allows for targeted and controlled delivery of an agent or agents to target site, such as specific cells or a specific tissue of an individual. An exemplary Agent Delivery System includes a platform, having a silica crystal lattice base plate, coated with a collagen-containing layer. At least one agent of interest is disposed within the platform, for delivering to the target site. The agent is released when the Agent Delivery Platform is exposed to collagen-degrading compounds. Collagen-degrading compounds can be secreted by cells within a target site.

### CROSS-REFERENCE TO RELATED APPLICATIONS

This application claims priority from U.S. Provisional Application Ser. No. 60/763,011 filed Jan. 27, 2006, the entire disclosure of which is incorporated herein by this reference.

### STATEMENT REGARDING SPONSORED RESEARCH

This invention was made with government support under, grant number NAG2-1647 awarded by the National Aeronautics and Space Administration (NASA), and grant number 0508211 awarded by the National Science Foundation. The United States government has certain rights in the invention.

### BACKGROUND OF THE INVENTION

There are various obstacle to realizing the therapeutic potential of certain agents. Obstacles are often related to providing appropriately targeted and controlled delivery of the agents. For example, a considerable obstacle to the therapeutic use of small interfering RNAs (siRNAs), exogenous micro-RNAs (miRNAs), and complimentary micro-RNAs (c-miRNAs) and antagomers is providing controlled, precision delivery.

Various systems and devices have been attempted for use in delivery applications, but have certain limitations. Some devices lack precision, for example, due to their bulky nature. Certain nanoparticles have been used in biomedical delivery applications, such as use in the area of drug delivery via cell membrane permeation. For example, gold microparticles coated with DNA have been introduced into cells by high-velocity acceleration to penetrate the cell membrane. (Yang, et al. (1990)). However, this method is problematic due to difficulties in controlling either the precise amount of DNA or the precise cell penetration modality.

Other nanostructures have been created for use in delivery applications, but have various limitations. For example, certain structures are not biocompatible, can produce toxic effects in target cells, can cause infections, or can produce other undesired immune reactions. Other structures lack the ability to provide for controlled delivery, instead supplying an overly rapid delivery of agents to cells, which can result in harm to the cells.

Accordingly, there remains a need in the art for a system for targeted and controlled delivery of an agent of interest to cells of an individual.

### BRIEF SUMMARY OF THE INVENTION

The invention meets the above-identified needs by providing a system and method for targeted and controlled delivery of an agent of interest to a target site of an individual.

The present invention includes Agent Delivery Systems, Agent Delivery Platforms, and methods for generating and using the Agent Delivery Systems and Platforms to deliver agents to target sites in a targeted and controlled manner.

An exemplary Agent Delivery System includes: an Agent Delivery Platform, including a base layer, and a collagen-containing layer coating the base layer. At least one agent for delivering to a target site is disposed within the Agent Delivery Platform, and the agent is released when the Agent Delivery Platform is exposed to collagen-degrading compounds.

In certain embodiments, the Agent Delivery Platform includes one or more additional layers coating the base layer. An exemplary Agent Delivery Platform includes a first collagen-containing layer, and a second collagen-containing layer, which can have a different concentration of collagen than the first collagen-containing layer.

In certain embodiments, the base layer is a crystal lattice, including silica spheres arranged in a face-centered cubic (FCC) lattice structure. In certain embodiments, the base layer is a crystal lattice, created using polymer colloids, such as polystyrene, parylene, or urethane colloids. In certain embodiments the base layer is an inverse replica of a crystal lattice, e.g., inverse replica of an FCC lattice structure.

An exemplary Agent Delivery System of the present invention includes: an Agent Delivery Platform, including, a base layer, and a collagen-containing layer coating the base layer; wherein at least one agent is disposed within the Agent Delivery Platform, wherein the collagen-containing layer degrades as a function of time when placed adjacent cells of a target site, such that the one or more agents are delivered to the target site in a targeted and controlled manner.

The Agent Delivery Platform has a three-dimensional porous structure, provided by the lattice, or inverse replica of the lattice, which as the solid base layer to support the collagen layer. The porous nature of the three-dimensional structure provides for an increased surface area on which the agent-containing collagen layer is presented. The increased surface area, e.g., relative to a flat surface, provides greater access to the agent-containing layer or layers of the Platform.

Without wishing to be bound by theory, it is believed that the Agent Delivery System delivers agent to the target site in a targeted and controlled manner when placed adjacent cells of the target site because the cells secrete collagenase, and other enzymes, that degrade the collagen layer over a period of time, releasing the agent to the target site in a gradual manner.

An exemplary Agent Delivery System of the present invention can be used in the following manner. A target site is identified on an individual to be treated. An Agent Delivery System is provided, which contains one or more agents selected to affect the target site in a desired manner. The Agent Delivery System is placed adjacent the target site, and affixed thereto for a period of time. The one or more agents are delivered to the target site as the cells secrete and transfer to the Agent Delivery System collagen-degrading enzymes. As the collagen degrades, the one or more agents are delivered to the target site.

The Agent Delivery Systems of the present invention are capable of delivering agents that are useful for a desired application. For example, the delivered agents can be intended for a therapeutic purpose, a diagnostic purpose, or another useful purpose. Examples of agents that can be delivered using the Agent Delivery Platform include: small molecules, proteins and peptides, nucleotides, gene-based agents, such as small interference RNAs (siRNAs) and micro RNAs (miRNAs), complementary miRNAs (c-miRNAs), antagomers, genes, viruses, viral constructs, antibodies, chemoattractants, cytokines, and other useful agents.

The Agent Delivery Systems allow for targeted delivery, in that an agent can be delivered to a specific target site, for example, specific cells or a specific tissue in an area of interest of an individual. Targeted delivery can be desirable for a number of reasons. For example, if a gene-based agent is being delivered, it can be targeted to very narrowly confined sites with focal pathology, such as injury sites, allowing treatment at the targeted site to occur, while avoiding systemic administration of the gene-based agent, which may create off-site side effects. Reducing or eliminating side effects on areas neighboring the targeted treatment site can be beneficial, for example, in cancer chemotherapy applications, or in wound healing applications involved in sustained burn injury.

For another example, targeted delivery can enhance bioavailability of the agent being delivered, allowing for treatment to be affected with lower doses of the agent. That is to say, lower doses of the agent are needed to affect treatment, because a greater amount of the administered agent is directed towards the targeted site.

The Agent Delivery Systems also allow for controlled delivery, in that the agents can be gradually delivered to cells. The focused and gradual delivery reduces or eliminates the need for large doses of agents that can be diluted by systemic administration, the need for repetitive administration of agents, and the risk of injury to cells from over-exposure to agents. The targeted and controlled delivered is also beneficial in economic terms, because it reduces or eliminates the need to repeatedly administer expensive therapeutic agents.

An exemplary method of generating an Agent Delivery Platform and System in accordance with the present invention, includes: preparing a base layer; providing collagen; providing an agent of interest for delivering to a target site; and coating the base layer with the collagen and the agent of interest. The base layer of an exemplary Agent Delivery Platform can be prepared in the following manner. A colloidal crystal lattice is created using colloids, or uniformly sized spheres of silica, polystyrene, biocompatible polymers, such as parylene, or urethane, or other acceptable material. Colloidal crystals are grown, and resulting deposits of colloidal crystals are collected and sintered. The resulting crystal lattice can be sized as desired.

The crystal lattice, itself, serves as the base layer in certain embodiments. In other embodiments, an inverse replica of the crystal lattice is constructed to serve as the base layer. An inverse replica of the template colloidal crystal lattice is generated using a resin. For example, an inverse carbon replica of the template can be generated using a phenolic resin, such as Furfuryl Alcohol (2-Furanmethanol) or Furcarb LP-520. The template colloidal crystal lattice can be removed, leaving the inverse replica base layer. For example, when silica colloids are used, the silicon dioxide spheres of the template crystal lattice can be dissolved in 2% hydrofluoric acid, leaving inverse replica base layer.

Collagen is provided in liquid form. An agent of interest is provided and added to the collagen mixture. In certain embodiments, multiple agents are added to the collagen mixture. The base layer is coated with the collagen and agent by immersing the base layer in the collagen-agent mixture, and incubating to form a collagen gel. The resulting Agent Delivery System, including the Agent Delivery Platform infused with the agent of interest, can be used to deliver the agent to a target site, e.g. specific cells.

### BRIEF DESCRIPTION OF THE DRAWINGS

FIG. 1 is a flow chart illustrating the steps involved in an exemplary method of making an Agent Delivery Platform and System in accordance with the present invention;

FIG. 2A is a scanning electron microscopic image of an exemplary base layer of an Agent Delivery Platform made in accordance with the present invention;

FIG. 2B is a scanning electron microscopic image of an exemplary Agent Delivery Platform made in accordance with the present invention;

FIG. 3 is a flow chart illustrating the steps involved in another exemplary method of making an Agent Delivery Platform and System in accordance with the present invention;

FIG. 4 is a graphic representation a study used to examine the efficacy of embodiments of the present invention, where Agent Delivery Platforms were placed adjacent a target subpopulation of cells, removed after a period of time, and the cells were observed under a fluorescent microscope for cell-associated cytoplasmic fluorescence intensity, to substantiate successful infection;

FIG. 5A includes a pair of pictures of cells in a target subpopulation to which agents were delivered using an Agent Delivery Platform of the present invention, showing infection-associated fluorescence activity, wherein the first panel is a phase-contrast image, while the second panel is a fluorescence image of the same field;

FIG. 5B includes a pair of pictures of cells neighboring target subpopulation to which agents were delivered using an Agent Delivery Platform of the present invention, wherein the viruses are not transferred and the neighboring cells, as evidenced by minimum fluorescence, wherein the first panel is a phase-contrast image, while the second panel is a fluorescence image of the same field;

FIG. 6A includes a pair of pictures of control cells, incubated with an Agent Delivery Platform without lentivirus, which show no fluorescence, wherein the first panel is a phase-contrast image, while the second panel is a fluorescence image of the same field;

FIG. 6B includes a pair of pictures of control cells, incubated with an Agent Delivery Platform with the lentiviral vector alone, without the luciferase and green fluorescence protein constructs, wherein the first panel is a phase-contrast image, while the second panel is a fluorescence image of the same field;

FIG. 6C includes a pair of pictures of cells treated with viral vector alone, not delivered using the Agent Delivery Platform, which cells undergo slow apoptosis, and die, wherein the first panel is a phase-contrast image, while the second panel is a fluorescence image of the same field;

FIG. 6D includes a pair of pictures of cells treated directly with lenti-luciferase, which cells undergo slow apoptosis, and die, wherein the first panel is a phase-contrast image, while the second panel is a fluorescence image of the same field;

FIG. 7A includes the results of a Western blot with anti-collagenase-1 antibody, where medium free of fetal bovine serum was incubated with cells and collected at various time points to determine the levels of secreted collagenase;

FIG. 7B includes pictures of cells infected with medium incubated with the Agent Delivery Platform (upper panels), and pictures of cells infected with medium incubated without Agent Delivery Platforms (lower panels), wherein cells infected with the medium showed no fluorescence, demonstrating that the collagenase enzyme released by the cells into the medium released the viral vector disposed in the collagen, wherein the right panels are phase-contrast images, while the left panels are fluorescence images of the same fields;

FIG. 8A includes the results of a Western blot with caspase-3 and caspase-3 antibodies, where cells were not infected or were invented by lentiviruses, wherein caspase activity increases with time; and

FIG. 8B includes the results of a cell viability assay, quantifying the cytotoxic level of the cells, where the percentage of cells dying increased with time.

### DESCRIPTION OF EXEMPLARY EMBODIMENTS OF THE INVENTION

The present invention includes systems and methods for targeted and controlled delivery of agents to target sites.

The present invention includes Agent Delivery Systems, Agent Delivery Platforms, and methods for generating and using the Agent Delivery Systems and Platforms to deliver agents to target sites of individuals in a targeted and controlled manner.

Agent Delivery Systems made in accordance with the present invention are biocompatible and have a porous, three-dimensional lattice structure, with a large surface area to volume ratio.

The Agent Delivery Systems can be used to deliver an agent to a target site in an individual, such as a human, a horse, a cow, a pig, a dog, a cat, a rabbit, a rat, a mouse, or another mammal.

The Agent Delivery Systems of the present invention are capable of delivering agents that are useful for a desired application. For example, the delivered agents can be intended for a therapeutic purpose, a diagnostic purpose, or another useful purpose. Examples of agents that can be delivered using the Agent Delivery Platform include: small molecules, proteins and peptides, nucleotides, gene-based agents, such as small interference RNAs (siRNAs) and micro RNAs (miRNAs), antagomers, genes, viruses, antibodies, chemoattractants, cytokines, and other useful agents.

The Agent Delivery Systems allow for targeted delivery, in that an agent can be delivered to a specific target site, for example, specific cells or a specific tissue in an area of interest of an individual. Targeted delivery can be desirable for a number of reasons. For example, if a gene-based agent is being delivered, it can be targeted to very narrowly confined sites with focal pathology, such as injury sites, allowing treatment at the targeted site to occur, while avoiding systemic administration of the gene-based agent, which may create off-site side effects. Reducing or eliminating side effects on areas neighboring the targeted treatment site can be beneficial, for example, in cancer chemotherapy applications, or in wound healing applications involved in sustained burn injury.

For another example, targeted delivery can enhance bioavailability of the agent being delivered, allowing for treatment to be affected with lower doses of the agent. That is to say, lower doses of the agent are needed to affect treatment, because a greater amount of the administered agent is directed towards the targeted site.

The Agent Delivery Systems also allow for controlled delivery, in that the agents can be gradually delivered to cells. The focused and gradual delivery reduces or eliminates the need for large doses of agents that can be diluted by systemic administration, the need for repetitive administration of agents, and the risk of injury to cells from over-exposure to agents. The targeted and controlled delivered is also beneficial in economic terms, because it reduces or eliminates the need to repeatedly administer expensive therapeutic agents.

An exemplary application of the present invention is in the treatment of an individual suffering from hair loss. An Agent Delivery Platform enriched with agents that will promote hair growth could be placed on the scalp of the individual for a period of time. The agents would be precisely delivered to cells on the scalp of the individual, but would not, for example, be delivered to cells on the forehead of the individual. The nano-structure of the Agent Delivery Platform makes it unobtrusive and discrete in appearance. This is just one example of an application in which the Agent Delivery Platform could be used. Other examples include, for example, wound healing, cancer treatment, and other desirable applications in which precise delivery of agents to specific sites is desired.

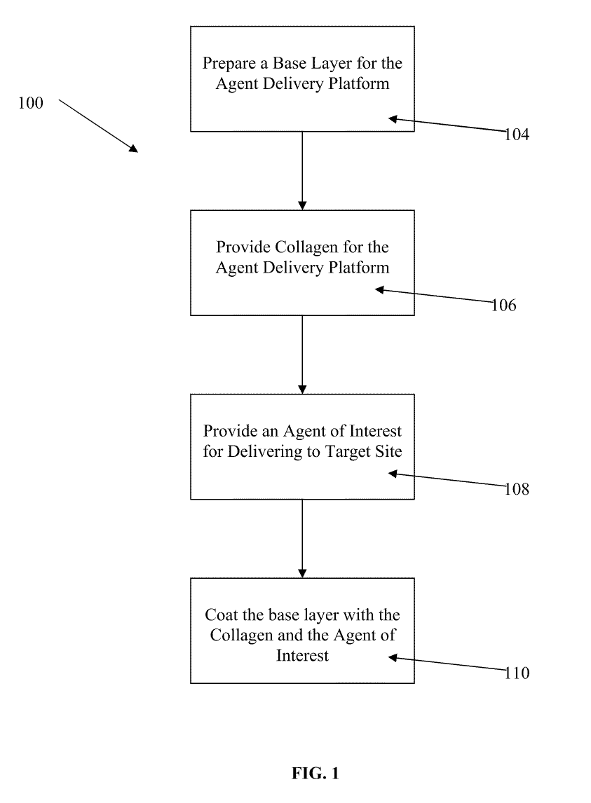

With reference to FIG. 1, an exemplary method 100 of generating an Agent Delivery Platform and System in accordance with the present invention, includes: preparing a base layer 104; providing collagen 106; providing an agent of interest for delivering to a target site 108; and coating the base layer with the collagen and the agent of interest 110.

The base layer of an exemplary Agent Delivery Platform can be prepared in the following manner 104. A colloidal crystal lattice is created using colloids, or uniformly sized spheres of silica, polystyrene, biocompatible polymers, such as parylene, or urethane, or other acceptable material. Such colloids may be purchased, for example, from Fiber Optic Center, Inc., (New Bedford, Mass.). Colloidal crystals can be grown, for example, using a slow crystallization process of monodispersed aqueous silica colloids in a glass cylinder. An exemplary process of growing such colloidal crystals can be conducted at ambient temperatures and can take about 10 months.

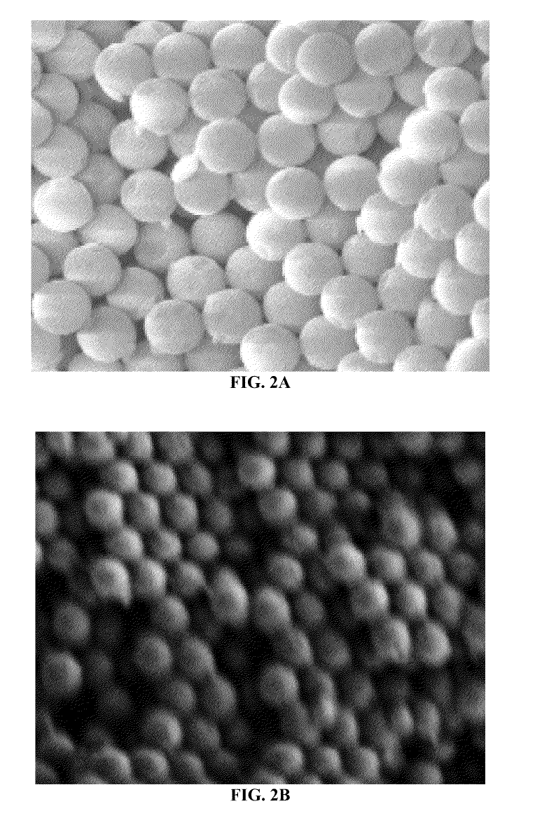

Resulting deposits of colloidal crystals are collected and sintered. Sintering can be conducted for about 2 to 8 hours at between about 700 and about 750° C., when silica is being used. The temperature selected should be below the melting point of the material of the template colloidal crystals. The sintering step results in a crystal lattice, including interconnected spheres. For example, when silica colloids are used, crystals obtained include interconnected silicon dioxide spheres arranged in a face-centered cubic (FCC) lattice structure. FIG. 2A is scanning electron microscopic image of such a crystal lattice, which includes interconnected silicon dioxide spheres arranged in an FCC lattice structure. The resulting crystal lattice can be sized as desired. For example, the crystals can be cut with a diamond saw and polished to a desired thickness, e.g., 1 mm.

Collagen is provided in liquid form 106. Various types of collagen are appropriate for use with the present invention, including, rat tail or bovine collagen. Appropriate collagen can be obtained, for example, from B.D. Chemical, Inc. (Salinas, Calif.). Collagen is in liquid form at low temperatures, e.g., about 4° C. The collagen can be mixed, for example, with acetic acid and provided at a desired concentration. The collagen can be further mixed with buffers, to produce a final desired collagen concentration, which is within a bioacceptable pH range, e.g., about 6.5 to about 7.5.

An agent of interest is provided 108. Agents can include, for example, small molecules, proteins and peptides, nucleotides, gene-based agents, such as small interference RNAs (siRNAs) and micro RNAs (miRNAs), antagomers, genes, viruses, antibodies, chemoattractants, cytokines, and other useful agents that are desired to be delivered to a target site in a targeted and controlled manner. The agent is added to the collagen mixture to yield a desired final concentration. In certain embodiments, it can be desirable to provide more than one agent, in which case, each of multiple agents can be added to the collagen mixture to yield a desired final concentration of each agent.

The crystal lattice base layer is sterilized and coated with the collagen and agent 110 by immersing the crystal lattice base layer in the collagen-agent mixture, and incubating at about 37° C. to form a collagen gel. The resulting Agent Delivery System, including the Agent Delivery Platform infused with the agent of interest, can be used to deliver the agent to a target site, e.g. specific cells. FIG. 2B is a scanning electron microscopic image of an exemplary Agent Delivery Platform, including a silica crystal lattice base layer, and a collagen layer.

In certain embodiments, it can be desirable to provide an Agent Delivery Platform that has not yet been infused with an agent. For example, it can be desirable to provide the base layer, coated with collagen, resulting in an Agent Delivery Platform. Before the Agent Delivery Platform is used to deliver at least one agent to a targeted site, the Agent Delivery Platform can be infused with the agent or agents. For example, the agent or agents could be provided in a collagen-agent mixture, into which the Agent Delivery Platform is immersed. For another example, the at least one agent could be injected or otherwise disposed within the collagen.

With reference again to FIG. 1, another exemplary method 100 of generating an Agent Delivery Platform and System in accordance with the present invention will now be described. A template colloidal crystal lattice is created using template colloids 102. As described above, colloidal crystals are grown, collected, and sintered, to yield a template crystal lattice, including interconnected spheres. For example, with reference to FIG. 2A, when silica colloids are used, template crystals obtained include interconnected silicon dioxide spheres arranged in an FCC lattice structure. The resulting template crystal lattice can be sized as desired.

An inverse replica of the template colloidal crystal lattice is generated using a resin. For example, an inverse carbon replica of the template can be generated using the phenolic resin known as Furfuryl Alcohol (2-Furanmethanol) or Furcarb LP-520, which can be obtained from Great Lake Chemical Corporation (a Chemtura Corporation Company, Middlebury, Conn.), or QO Chemical Inc. (Barrington, Ill.). More information about such resins can be found in Zakhidov, et. al. Science (1998), which is incorporated herein by this reference. A synthetic route can be used, which is similar to the one described in Zakhidov, et al., Science (1998). The template colloidal crystal lattice is removed, leaving the inverse replica base layer. For example, when silica colloids are used, the silicon dioxide spheres of the template crystal lattice can be dissolved in 2% hydrofluoric acid, leaving inverse replica base layer.

Collagen is provided in liquid form at a desired concentration 106. An agent of interest is provided 108. The agent is added to the collagen mixture to yield a desired final concentration. In certain embodiments, it can be desirable to provide more than one agent, in which case, each of multiple agents can be added to the collagen mixture to yield a desired final concentration of each agent.

The inverse replica base layer is sterilized and coated with the collagen and agent 110 by immersing the crystal lattice base layer in the collagen-agent mixture, and incubating to form a collagen gel. In such embodiments, the interstitial space within the inverse replica base layer is filled with the collagen-agent mixture, and allowed to solidify therein. The resulting Agent Delivery Platform can be used to deliver the agent to target sites.

In certain embodiments, it can be desirable to provide an Agent Delivery Platform that has not yet been infused with an agent. For example, it can be desirable to provide the base layer, coated with collagen, resulting in an Agent Delivery Platform. Before the Agent Delivery Platform is used to deliver at least one agent to a target site, the Agent Delivery Platform can be infused with the agent or agents. For example, the agent or agents could be provided in a collagen-agent mixture, into which the Agent Delivery Platform is immersed. For another example, the at least one agent could be injected or otherwise disposed within the collagen.

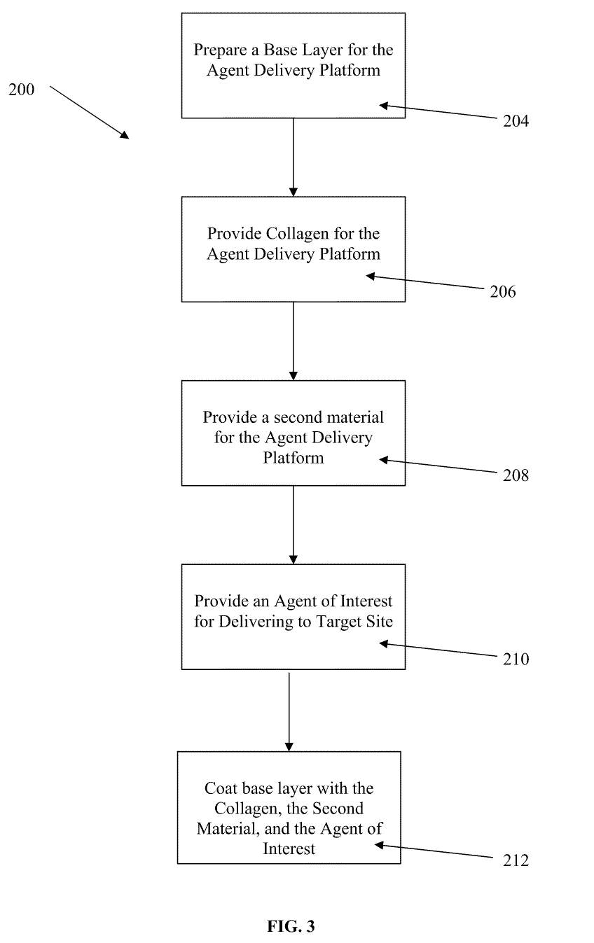

With reference to FIG. 3, another exemplary method 200 of generating an Agent Delivery Platform and System in accordance with the present invention, includes: preparing a base layer 204; providing collagen in a liquid form 206; providing a second material 208; providing an agent of interest for delivering to a target site 210; and coating the base layer with the collagen and the agent of interest 212.

The base layer of an exemplary Agent Delivery Platform can be prepared in the following manner 204. A colloidal crystal lattice is created using colloids, or uniformly sized spheres of silica, polystyrene, a biocompatible polymer, such as parylene, or urethane, or other acceptable material. Colloidal crystals can be grown, for example, using a slow crystallization process of monodispersed aqueous silica colloids in a glass cylinder. An exemplary process of growing such colloidal crystals can be conducted at ambient temperatures and can take about 10 months.

Resulting deposits of colloidal crystals are collected and sintered. Sintering can be conducted for about 2 to 8 hours at between about 700 and about 750° C., when silica is being used. The temperature selected should be below the melting point of the material of the template colloidal crystals. The sintering step results in a crystal lattice, including interconnected spheres. For example, when silica colloids are used, crystals obtained include interconnected silicon dioxide spheres arranged in a face-centered cubic (FCC) lattice structure. FIG. 2A is scanning electron microscopic image of such a crystal lattice, which includes interconnected silicon dioxide spheres arranged in an FCC lattice structure. The resulting crystal lattice can be sized as desired. For example, the crystals can be cut with a diamond saw and polished to a desired thickness, e.g., 1 mm.

An alternative base layer of an exemplary Agent Delivery Platform can be prepared in the following manner 204. An inverse replica of the colloidal crystal lattice can be generated. For example, a carbon-based or polymer-based inverse replica base layer can be generated and the silica crystal lattice can be dissolved, as described above.

Collagen is provided in liquid form at a desired concentration 206. A second material is provided in liquid form 208. The second material could be, for example, collagen provided at a second desired concentration, which differs for the concentration of the first provided collagen. In other embodiments, the second material could be actin, solubilized in high salt solution, In other embodiments the second material could be vimentin, first solubilized in 2M urea, then diluted with phosphate buffered saline. In other embodiments, the second material could be fibronectin, laminin, or proteoglycan, solubilized in phosphate buffered saline.

The collagen and the second material are bioacceptable materials and/or materials that are absorbable by the body of an individual being treated. For example, the collagen could be rat tail or bovine collagen, and the second material could be rat tail or bovine collagen, actin, vimentin, fibronectin, laminin, or proteoglycan. Protein can be isolated from non-muscle sources to ensure biocompatibility with certain target cell populations. Bioacceptable and absorbable materials are materials that create minimal toxic effects, which toxic effects include inflammatory response beyond a typical repair and injury response, free-radical generation from the cells, and increased localized cell death.

The base layer can be coated 212 with the collagen by immersing the base layer in the collagen, and allowing the collagen to solidify. The second material can be added, creating an interlocking three-dimensional junction between the two materials. For example, when the second material is soluble monomeric actin, it can polymerize under changing salt concentrations, and precipitate to form a final collagen/protein three-dimensional interconnecting structure.

The agent of interest is provided 210. The agent can be added by mixing it with the collagen or the second material, before the base layer is coated with the collagen and the second material. In certain embodiments, it can be desirable to provide more than one agent with the Agent Delivery System, in which case, each of multiple agents can be added to the collagen mixture and/or to the second material mixture to yield a desired final concentration of each agent. In certain embodiments a first agent, or group of agents, can be added to the collagen mixture, while a second agent, or group of agents, can be added to the mixture of the second material. For example, in embodiments including two layers of collagen, the base layer can be coated with a first concentration of collagen mixed with a first agent of interest, and a second concentration of collagen mixed with a second agent of interest.

In certain embodiments, it can be desirable to provide an Agent Delivery Platform that has not yet been infused with an agent. For example, it can be desirable to provide the base layer, coated with collagen and a second material, resulting in an Agent Delivery Platform. Before the Agent Delivery Platform is used to deliver at least one agent to a targeted site, the Agent Delivery Platform can be infused with the agent or agents. For example, the agent or agents could be provided in another collagen-agent mixture, into which the Agent Delivery Platform is immersed. For another example, the agent or agents could be injected or otherwise disposed within the collagen and second material layers.

An exemplary Agent Delivery System includes: an Agent Delivery Platform, including a base layer, and a collagen-containing layer coating the base layer. At least one agent for delivering to a target site is disposed within the Agent Delivery Platform, and the agent is released when the Agent Delivery Platform is exposed to collagen-degrading compounds. In certain embodiments, the collagen-containing layer includes a second material, such as actin, vimentin, fibronectin, laminin, or proteoglycan, where interlocking three-dimensional junctions are provided between the collagen and the second material. In certain embodiments, the Agent Delivery Platform includes one or more additional layers coating the base layer. Layer can be composed of collagen; another material, such as actin, vimentin, fibronectin, laminin, or proteoglycan; or a layer including collagen and a second material, where interlocking three-dimensional junctions are provided between the collagen and the second material.

In certain embodiments, the Agent Delivery Platform includes a first collagen layer, and a second collagen layer, which can be of a different concentration than that of the first collagen layer. In certain embodiments, the base layer is a crystal lattice, including silica spheres arranged in an FCC lattice structure. In certain embodiments the base layer is an inverse replica of a crystal lattice, including silica spheres arranged in an FCC lattice structure.

An exemplary Agent Delivery System of the present invention includes: an Agent Delivery Platform, including, a base layer, and a collagen layer, coating the base layer; wherein one or more agents are disposed within the collagen layer; wherein the collagen layer degrades as a function of time when placed adjacent cells of a target site, such that the one or more agents are delivered to the target site in a targeted and controlled manner. The Agent Delivery Platform has a three-dimensional porous structure, provided by the lattice, or inverse replica of the lattice, which as the solid base layer to support the collagen layer. The porous nature of the three-dimensional structure provides for an increased surface area on which the agent-containing collagen layer is presented. The increased surface area, e.g., relative to a flat surface, provides greater access to the agent-containing collagen layer.

Without wishing to be bound by theory, it is believed that the Agent Delivery System delivers agent to the target site in a targeted and controlled manner when placed adjacent cells of the target site because the cells secrete collagenase, and other enzymes, that degrade the collagen layer over a period of time, releasing the agent to the target site in a gradual manner. It is contemplated that the rate of release of agents disposed in an Agent Delivery Platform can be varied. For example, the surface area on which the agent-containing collagen layer is presented can be varied. For another example, an exemplary Agent Delivery System could include exogenous collagenase, or other enzyme, that can assist with the gradual degradation of the agent-containing layer.

In is contemplated that members of the matrix metalloproteinase family of proteinases (MMPs) can assist with the process of releasing agents from an Agent Delivery Platform of the present invention. Such MMPs include: collagenases (MMP-1, 8, 13, 18), gelatinases (MMP-2, gelatinase A; MMP-9, gelatinase B); stromelysins (MMP-3, 10, 11), matrilysins (MMP-7, 26), membrane-type MMPs (MMP-14, 15, 16, 17, 24, 25), and other MMPs (e.g., MMP-12, 19, 20, 21, 23, 28). Such MMPs are synthesized by many cell types. Additionally, exogenous MMPs may be provided as part of an exemplary Agent Delivery System of the present invention for use in varying the release rate of the agents disposed in the Agent Delivery Platform.

An exemplary Agent Delivery System of the present invention can be used in the following manner. A target site is identified on an individual to be treated. An Agent Delivery Platform is provided, which contains one or more agents selected to affect the target site in a desired manner. The Agent Delivery Platform is placed adjacent the target site, and affixed thereto for a period of time. The Agent Delivery Platform can be affixed to the target site, for example, by covering the placed Agent Delivery Platform with a traditional bandage, e.g. adhesive bandage, wrapped gauze bandage, etc. The Agent Delivery Platform is removed after a desired period of time, i.e., after a desired amount of the one or more agents have been delivered to the target site in a targeted and controlled manner. The one or more agents are delivered to the target site as the cells secrete and transfer to the Agent Delivery Platform collagen-degrading enzymes. As the collagen degrades, the one or more agents are delivered to the target site.

The system can also include one or more exogenous enzymes useful for increasing the rate of delivery of the one or more agents to the target site. For example, exogenous collagenase could be provided. In this regard, an exemplary Agent Delivery System of the present invention could include an Agent Delivery Platform, and a bandage infused with exogenous collogenase.

In an exemplary embodiment, an Agent Delivery System includes a base layer that has been immersed in a first concentration of collagen in liquid form, containing a first agent of interest, and incubated to allow the collagen containing the first agent to gel. The base layer and first collagen-agent layer are then be immersed in a second concentration of collagen in liquid form, containing a second agent of interest, and incubated to allow the collagen containing the second agent to gel. The resulting Agent Delivery System includes two layers of collagen containing two different agents of interest. When the exemplary System is used to deliver the agents to a target site, the second agent, being disposed in the outside layer, is the first to be delivered to the target site. The first agent, which becomes available as the outer layer of collagen dissolves and exposes the inner layer of collagen, is then delivered to the target site.

It is contemplated that various collagen-containing layers and agent-and-collagen-containing layers could be provided to allow for delivery of various agents over a period of time. In one example, a collagen-containing layer could be disposed between two agent-and-collagen-containing layers, such that there is a delay between the delivery of the agent contained in the outer agent-and-collagen-containing layer, and the delivery of the agent contained in the inner agent-and-collagen-containing layer. It is contemplated that a biocompatible detection agent could be disposed within an inner collagen-containing layer, such that it could be easily determined when the agents contained in the outer layers had been delivered, signaling, for example, an appropriate time to remove the Agent Delivery System.

The Agent Delivery Platform can take on a variety of shapes and sizes. For example, an exemplary Agent Delivery Platform can be shaped such that it can be placed over a target site. For example, if the target site is a wound at the tip of a finger of an individual, the Agent Delivery Platform could be provided in the shape of a finger tip.

The present invention is further illustrated by the following specific but non-limiting examples. The following examples may include compilations of data that are representative of data gathered at various times during the course of development and experimentation related to the present invention.

### EXAMPLES

### Methods

Colloidal crystals were grown using a slow crystallization process of monodispersed aqueous silica colloids in a glass cylinder, typically for 10 months at ambient temperature. Zakhidov (1998). Resulting deposits were polycrystalline with rod-like single crystals, which were sintered for several hours at 700-750° C. With reference to FIG. 2A, the crystals thus obtained were closely packed, interconnected silicon dioxide spheres arranged in an FCC lattice structure. The diameter of such spheres can range from about 20 nm to about 10 μm, with interconnected voids between the spheres; the silica colloids used in the studies described herein had sphere diameters between about 200 and about 400 nm. The crystals were cut using a low-speed diamond saw, and polished to a thickness of less than a millimeter, to ensure that when tested, they would be suspended above monolayer cultures. Inverse carbon replicas of the colloidal crystals were fabricated using the phenolic resin route (Zakhidov (1998)), followed by dissolution of the silica spheres in 2% HF.

The following antibodies were obtained. Peroxidase-conjugated sheep immunoglobulin G (IGG) fraction to Rabbit IGG was obtained from MP Biomedicals (Solon, Ohio), and goat anti-mouse IgG, peroxidase conjugated, from Pierce Biotechnology (Rockford, Ill.). Monoclonal mouse matrix metalloprotease-1 (MMP-1) (i.e. collagenase-1) antibody was obtained came from Lab Vision Corporation (Fremont, Calif.), rabbit polyclonal to active caspase 3 from Abcam (Cambridge, Mass.), and polyclonal rabbit caspase-3 and β-actin antibody were purchased from Santa Cruz Biotechnology (Santa Cruz, Calif.).

Human lung embryonic fibroblasts (WI-38 cell strain) were cultured in minimum essential medium (MEM) (Hyclone, Logan, Utah) supplemented with 10% heat-inactivated fetal bovine serum (Hyclone, Logan, Utah); cells at early passages (PDL 30-35) were used for the studies described herein.

The viral constructs were packaged in VSV-G pseudoviral particles using feline immunodeficiency virus (FIV) vectors (System Biosciences, Mountain View, Calif.), and transfected to packaging cells by Lipofectamine 2000 (Invitrogen, Carlsbad, Calif.), according to the manufacturer's specifications. Lenti-luciferase virus was made from the package of this gene construct (System Biosciences, Mountain View, Calif.) using the 293-T17 culture system. Two milliliters of the culture supernatant containing the packaged lenti-luciferase viral particles were used to infect WI-38 fibroblast cultures in 35 mm glass-bottomed culture dishes, maintained for observation for 96 hrs in a humidified atmosphere containing 5% CO₂ at 37° C.

Colloidal crystals were rinsed in 70% ethyl alcohol, and exposed to UV light overnight inside the cell culture hood for sterilization. All procedures were performed on ice under sterile conditions. Rat-tail collagen type I was used for the experiment, 4.79 mg/ml mixed with acetic acid (0.02 N) to a final concentration of 2.85 mg/ml. This collagen stock was mixed with the vehicle buffer (NaCl (0.75 M) and 50 mM phosphate-buffered saline (PBS) in 50 ml of water) and neutralization buffer (NaHCO₃ (260 mM) with 4-(2-hydroxyethyl)-1-piperazine ethanesulfonic acid (HEPES) (200 mM) and NaOH (50 mM) in 50 ml of water) in the ratio 7:2:1, to produce a final collagen concentration of 2 mg/ml. pH was confirmed at 7.00 using pH paper.

Polybrene (Sigma, St. Louis, Mo.) was added to the lentivirus to a final concentration of 5 mg/ml, which was then added to the collagen in the ratio of 15:100 μl, and 50 μl of the collagen-virus mixture was micropipetted into Petri dishes. The sterilized colloidal crystals were slowly immersed in the collagen-virus mixture, and incubated at 37° C. for 30 min-1 hr to form a collagen gel. The crystalline samples, coated with collagen and virus, were transferred onto monolayer cultures in glass-bottom Petri dishes. The cells with the colloid collagen virus, i.e. the cells exposed to the Agent Delivery Platform, were incubated at 37° C. for 24-96 hrs. After 96 hrs the Agent Delivery Platforms were removed, and the cells were observed under the fluorescent microscope to determine the infection level of the virus.

A scanning electron microscope was used in the studies described herein. Imaging of colloidal crystals and colloidal crystals coated with collagen was carried out on a SUPRA™ 35VP Ultra-high performance VP field emission scanning electron microscope, at an accelerating voltage of 3-5 KV. The vacuum of the chamber was kept around 9×10⁻⁹ Torr. For Scanning Electron Microscopy (SEM) analysis, the colloidal crystalline samples were adhered onto sample stubs.

An immunofluorescence microscope was used in the studies described herein. WI-38 cells were plated in 35 mm glass-bottom Petri dishes (MatTek Corporation, Ashland, Mass.). Three days later, the cells were infected with virus delivered with or without the Agent Delivery Platform for 96 hrs, as described above. Cells were then fixed with 4% paraformaldehyde in PBS, and directly imaged with an Axiovert 200 M epi-fluorescence microscope (Carl Zeiss, Jena, Germany), using a 63×1.3 numerical aperture PlanApo objective. Figures were formatted using Adobe Photoshop (Adobe Systems, Mountain View, Calif.).

Western blot analysis was used in the studies described herein. Fibroblast cultures of the WI-38 strain with and without infection with the viral vector construct were collected with cell scrapers in the medium, along with ice-cold PBS, and centrifuged at 250 g for 5 min. Cell extracts were prepared in radioimmunoprecipitation (RIPA) buffer (20 mM tris (hydroxymethyl) aminomethane hydrochloride (Tris-HCl), pH 7.4, 300 mM sodium chloride (NaCl), 2% Triton, 2 mM ethylenediaminetetraacetic acid (EDTA), and 0.2% SDS) containing protease inhibitors (Calbiochem, San Diego, Calif.) with constant agitation at 4° C. for 30 min, followed by centrifugation at 15,000 g for 20 min. The cellular polypeptides from the protein extract were released by boiling in double-strength Laemmli SDS-sampling buffer at 95° C. for 5 min. Equal amounts of proteins were separated on (9-12%) sodium dodecyl sulfate polyacrylamide gel electrophoresis (SDS-PAGE), and blotted onto a nitrocellulose membrane with a transfer buffer containing 25 mM Tris base, 192 mM glycine, and 20% methanol for 3.5 hr with a constant current of 325 mA. Nitrocellulose membranes were blocked in 5% non-fat dry milk (LabScientific, Livingston, N.J.) in TBST (20 mM Tris-HCl (pH 7.4), 500 mM NaCl, and 0.1% Tween-20). The blots were washed and incubated with indicated primary antibodies on a rotary shaker at 4° C. overnight. After washing, the blots were incubated with horseradish peroxidase (HRP)-conjugated second antibodies for 1 hr at room temperature, then with enhanced chemiluminescence reagent (ECL) (Amersham, Piscataway, N.J.) for 1 min, and visualized by exposure to Kodak X-OMAT film.

For collagenase immunoblots, media from cells incubated for different time intervals were collected. Protease inhibitors were added, and the samples were concentrated using centriplus centrifugal filter devices (Millipore, Billerica, Mass.), according to the manufacturer's instructions. The concentrated media were loaded onto 9% SDS-PAGE, as above.

Trypan blue exclusion assays were used to assess cell viability for the studies described herein. Cultures were processed initially to collect medium supernatant containing the detached cells, which were harvested by further centrifugation to generate cell pellets. They were then added to the fraction of attached cells, which were collected by treating the adherent remaining monolayer cultures from previous steps with 0.05% trypsin, 0.02% EDTA. The combined adherent and detached cell populations were then processed to determine cell viability in the cultures, by Trypan-blue exclusion assay. Trypan blue (Sigma, St. Louis, Mo.) was mixed with cells (1:1), and live cell indices were determined by counting cells that do not take up this dye manually on an inverted microscope.

Results.

The colloidal crystals were coated with collagen, a natural biopolymer, as a surfactant carrying the viral vector construct, as described above. With reference to FIG. 2A, SEM pictures of the colloid show a regularly spaced arrangement of voids within a colloidal crystal array. With reference to FIG. 2B, colloidal crystals coated with collagen show no significant changes in colloid morphology; the collagen seems to pack the voids of the colloidal crystal.

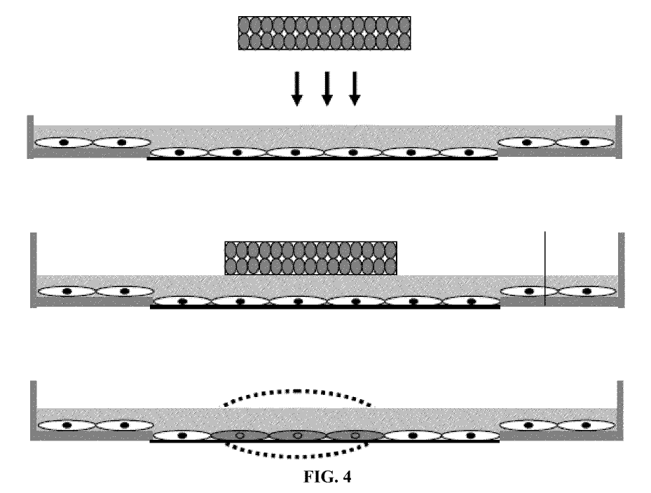

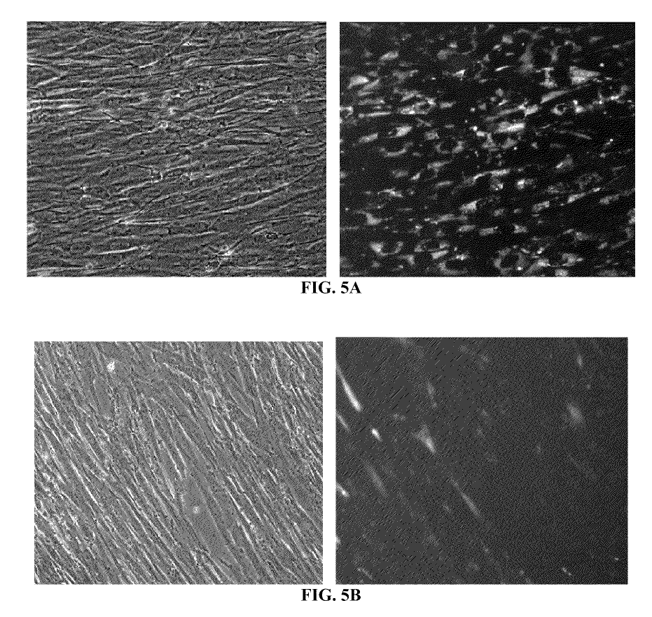

With reference to FIG. 4, the Agent Delivery Platform is placed over a target subpopulation of cells. The viral DNA is shown to be precisely transferred to a target subpopulation of cells in the monolayer culture. As described above, rat-tail collagen in solution containing the lentiviral luciferase construct is first added to the colloid in a precursor-soluble form at low temperature (4° C.); as the temperature rises to 37° C., the virus-containing liquid collagen gels, filling the voids of the nano-scale porous colloid, as shown in FIG. 5B. These Agent Delivery Platforms were then positioned above confluent cultures of WI-38 human fibroblasts; infection efficiency of luciferase gene expression was evaluated by fluorescence microscopy. With reference to FIGS. 5A and 5B, the colloid-collagen-lenti-luciferase infects specific subpopulations of confluent fibroblasts with close to 90% efficiency, while neighboring cells show very little infection-associated luciferase fluorescence activity. This result shows that after about 96 hours maximal detectable fluorescence intensity is reached, because the lenti-luciferase gene viral particles are released from the colloid-collagen-virus platforms before infecting the cells beneath them, and expressing gene-associated fluorescence. It is believed that this suspension prior to reaching maximum fluorescence activity could correlate with the period required for cells to secrete sufficient collagenase to digest the collagen, which encapsulates the viral particles.

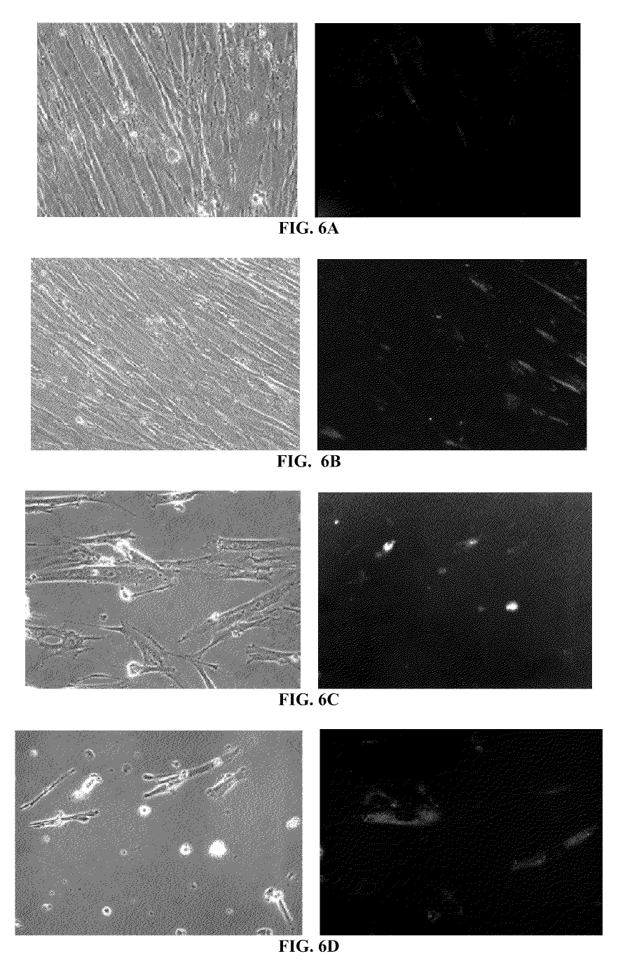

With reference to FIGS. 6A and 6B, negative controls of colloid-collagen-lentiviral vector alone, or colloid-collagen minus virus, show no fluorescence activity. With reference to FIGS. 6C and 6D, in sharp contrast, in control infections with either lentiviral vector alone at the same titre, or lenti-luciferase alone, uncoupled with Agent Delivery Platform, the infection kills most cells in the monolayer culture bearing the viral expression construct by 96 hrs; apoptotic death is observed to begin at 24 hrs post-infection, and reaches its maximal level at 96 hours. This result suggests that: the nanosized colloid beads are a feasible delivery device; and recipient fibroblast cultures secrete collagenase to digest the collagen slowly, since the maximal fluorescence intensity is only observed at 96 hours post-infection. Moreover, this slow release of the lentivirus allows infection of the cultures, without causing cell death, as shown by controls not using the colloidal crystal-collagen device.

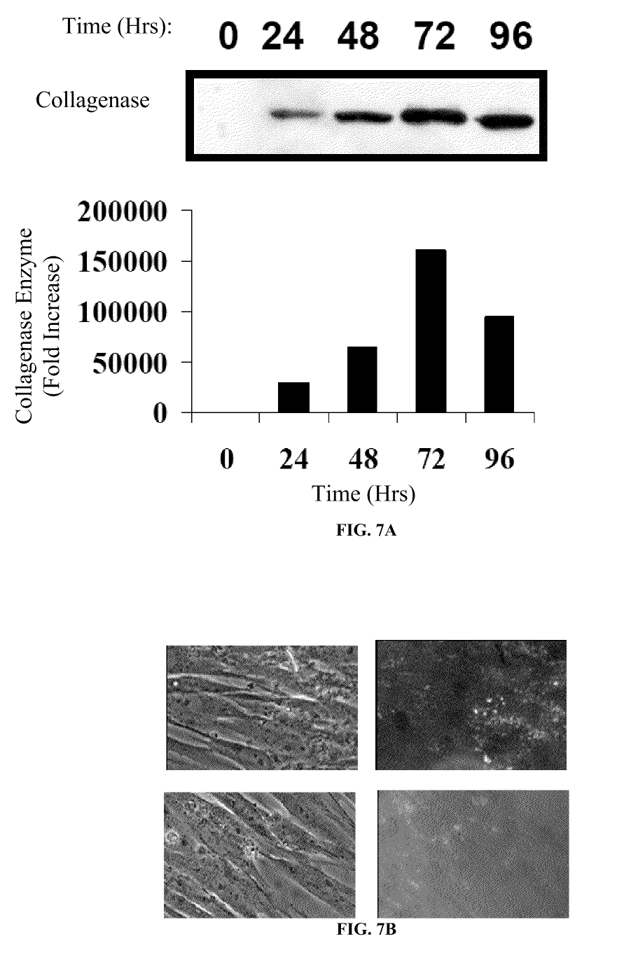

The plain MEM (without fetal bovine serum, or FBS) used for incubating the WI-38 cells was assayed for secretion of collagenase into the culture medium. Media were collected from cells at 24, 48, 72 and 96 hours; collagenase protein was quantified on Western blots, as shown in FIG. 7A. This experiment confirms the release of collagenase by cells. Chua (1985). To investigate whether collagenase is the means of releasing the viral particles, and not the culture medium itself, the Agent Delivery Platform is incubated in MEM medium containing 10% FBS and antibiotics for 96 hours, in the absence of cultured cell monolayer; colloidal crystals with collagen alone were used as a control. After 96 hrs, media from these colloidal crystals were used to infect the cells directly; 96 hrs after infection, the cells were observed in the fluorescent microscope. With reference to FIG. 7B, the study illustrates that the virus is not released from the collagen by incubation in cell-free, 10% FBS medium, even after 96 hrs. Thus, the release of the viral particles is due to the active participation of cell-secreted collagenase, rather than any biological activity of the fetal bovine serum in the culture medium.

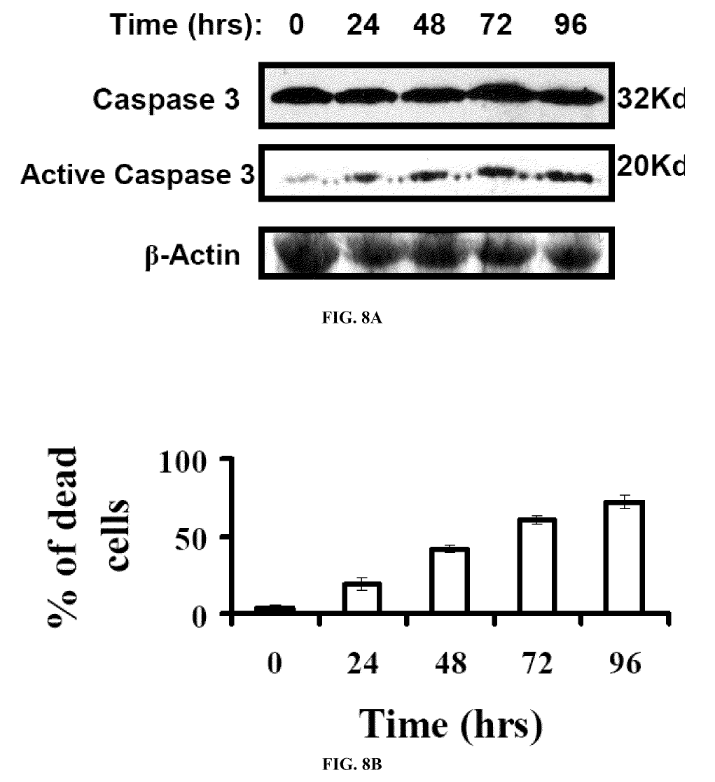

Caspase-3 blots and Trypan blue exclusion assay were used to establish that cell death after infection with virus directly, not embedded in the, is due to apoptosis. With reference to FIG. 8A, Caspase-3 activity was quantified using Western blots. Trypan blue staining was used to estimate the percentages of dead and live cells. With reference to FIG. 8B, by 96 hrs the percentage of dead cells was above 60%. Thus, it is shown that the Agent Delivery Platform provides not only precision focal delivery, but also the time-dependent release of infectious particles, to avoid rapid infection-induced cytopathic effects.

Discussion.

The studies described herein demonstrate the efficacy of the Agent Delivery Platform of the present invention for direct, precision delivery of agents to target sites in a controlled manner, i.e., designed such that the agent is gradually released over a period of time. As such, the Agent Delivery System of the present invention is a useful for a variety of delivery applications.

Embodiments of the system of the present invention are relatively inexpensive to produce, with a colloid substrate as the carrier to package the agent-of-interest, produces a complete, biocompatible delivery system. The efficacy studies described herein show that delivery is precise to a cell subpopulation immediately proximal to the Agent Delivery Platform. Additionally, the studies described herein show that the release of the viral particles is time-dependent. As such, in the case when viral particles are being delivered, for example, the cell death usually associated with high titer viral infection can be avoided.

The following contribute to the rationale for using collagen as the material to encapsulate the agent of interest. Collagen is readily cleaved by ubiquitous cell-associated collagenases; collagen is in liquid form at 4° C., allowing even suspension of the mixture with the agent of interest, e.g. viral particles, to form a coating on the colloidal crystal; and the coated agent-containing-collagen can be easily solidified by transfer to 37° C., thus firmly affixing it to the colloid crystal-backbone substratum. The entire set-up can then be easily used by placing the Agent Delivery Platform adjacent target cells.

Without wishing to be bound by theory, it is believed that the Agent Delivery Platform of the present invention operates in the following manner. With reference to FIG. 7A, the release of the viral particles from the encapsulated collagen matrix appears to be accomplished by collagenases secreted by the recipient cell cultures. These enzymes are metalloendopeptidases; it is believed that they mediate the initial and rate-limiting step in interstitial collagen degradation, by cleaving the three collagen polypeptide chains at a single locus three-fourths of the distance along the collagen molecule from its N-terminal end. (Gross, (1965)). Upon collagenase digestion, the collagen disintegrates and the embedded virus is thus released into the culture medium immediately adjacent to the target subpopulation of cells. With reference to FIG. 7B, the failure to release the viral particles in the context of cell-free fetal bovine serum-containing medium alone illustrates the role of target cell cooperation in delivery, by secreting the enzyme, collagenase.

Comparing cultures processed for lenti-luciferase viral infection with and without the colloid-collagen delivery system shows that, in the former case, the controlled release of the virus particles prevents the cytopathic effects seen in the latter case; this illustrates a further advantage of the Agent Delivery System of the present invention. In general, using lentiviral particles as part of an exemplary gene delivery system balances the need for sufficient dosage of infecting viral particles to induce effective actions by delivered gene expression, against the usual induction of cell death associated with virus infection. The results shown in FIG. 5D demonstrate that infection with lenti-luciferase viral particles directly, not embedded in the colloidal crystal-collagen delivery system, indeed causes cells to experience apoptotic death, evidenced by caspase activation, hallmarked by 10 kDa autocatalytic end products. (Thornberry, (1998)).

The present invention has various advantages over known technologies. The present invention includes an Agent Delivery Platform that is a biocompatible, porous matrix with an surface to volume ratio that is relatively large in various embodiments. There are other porous structures that can be produced; however, no previously described nanostructure has been successfully used in a delivery system for holding, and slowly releasing, agents into cells. Moreover, existing technologies providing porous structures all have intrinsic disadvantages.

For example, alumina templates, as described in Bauer, et at (2004), can be used to create substrates with high pore density and narrow pore size distribution. However, they do not have sufficient surface to volume ratio to deliver agents in a controlled manner. Studies conducted to compare efficacies of the Agent Delivery System of the present invention and the alumina template nanostructure for delivering viral particles show that use of the alumina template nanostructure generally results in cell death (data not shown). In other words, the alumina template nanostructure provide for an overly rapid delivery of agents to cells, not a controlled release and delivery of agents to cells.

Another nanostructure is a porous silicon structure, as described in Angelescu, et al. (2003). Porous silicon can be produced by non-uniform etching during the electropolishing of silicon with an electrolyte-containing hydrofluoric acid. The etching results in disordered pores with nanocrystals remaining in the inter-pore regions. Such nanocrystals are often not biocompatible, and can produce toxic effects in target cells. The porous silicon surface contains impurities from the air and the etching process that could affect biological systems, such as living cells, that are in contact with the surface. Common impurities include hydrogen, fluorine and oxygen. As much as 1% oxygen is normally adsorbed within minutes of air-drying. Over a few days Si—O—Si, O—Si—H and O₃—Si—H groups are formed. These oxides at the porous silicon surface and are thought to play a crucial role in limiting the biocompatibility of the material.

Block copolymers, as described in Pistel, et al. (1999), consist of regular arrays of two different types of polymers. By etching away one of the constituent polymers, it is possible to create a highly porous structure; but, such porous structures have not been used to create a porous substrate for drug delivery applications. Block copolymers have been used to create micro-spheres, which have been used for drug delivery applications. Devices based on bulk silicon semiconductors have been provided for in vitro biosensing applications; however, this form of silicon is not biocompatible and this incompatibility as thus far prevented its use in vivo. Bulk silicon-based integrated circuits need “packaging” in a biocompatible material if they are to be used in and linked to living tissues.

Microfabrication has been used to create complicated MEMS-based drug delivery devices, as described in Voskerician, et al. (2003). It might be possible to use microfabrication to create a porous substrate for drug delivery; however, this would require complicated fabrication techniques that would be difficult and expensive to implement. In any event, MEMS-based devices have a disadvantage of failing to be biocompatible. Such devices further have a disadvantage of being associated with biofouling, which refers to the negative impact of surrounding tissue on the function of a device. Such devices are also difficult to fabricate, and their extended use in vivo may cause infections or other undesired immune reactions.

Additionally, embodiments of the present invention have the advantage of requiring no complicated fabrication processes involving organic solvents, which are required by certain other polymer techniques. Moreover, embodiments of the base layer of the Agent Delivery Platform can be infiltrated with biocompatible polymers such as parylene, and urethane, which then can be used for the described precision delivery of the virus. For example, biocompatible polymers can be used in situations where it is determined that an adverse reaction to the silica colloids could be experienced at a target site.

One of ordinary skill in the art will recognize that modifications and variations are possible without departing from the teachings of the invention. This description, and particularly the specific details of the exemplary embodiments disclosed, is provided primarily for clearness of understanding and no unnecessary limitations are to be understood therefrom, for modifications and other embodiments will become evident to those skilled in the art upon reading this disclosure and may be made without departing from the spirit or scope of the claimed invention.

Unless otherwise indicated, all numbers expressing quantities of ingredients, properties such as reaction conditions, and so forth used in the Specification and Claims are to be understood as being modified in all instances by the term “about.” Accordingly, unless indicated to the contrary, the numerical parameters set forth in the Specification and Claims are approximations that can vary depending upon the desired properties sought to be determined by the present invention.

Notwithstanding that the numerical ranges and parameters setting forth the broad scope of the invention are approximations, the numerical values set forth in the example sections are reported as precisely as possible. Any numerical value, however, inherently contain certain errors necessarily resulting from the standard deviation found in their respective testing measurements.

Throughout this application, various publications are referenced. All such references are incorporated herein by reference, including the references set forth in the following list:
1. Huo, Q. et al. A new class of silica cross-linked micellar core-shell nanoparticles. J. Am. Chem. Soc. 128, 6447-6453 (2006).
2. Brannon-Peppas, L. & Blanchette, J. O. Nanoparticle and targeted systems for cancer therapy. Adv. Drug Del. Rev. 56, 1649-1659 (2004).
3. Ferrari, M. Cancer nanotechnology: opportunities and challenges. Nat. Rev. Cancer 5, 161-171 (2005).
4. Lasic, D. D. Doxorubicin in sterically stabilized liposomes. Nature 380, 561-562 (1996).
5. Medina, O. P., Zhu, Y. & Kairemo, K. Targeted liposomal drug delivery in cancer. Curr. Pharm. Des. 10, 2981-2989 (2004).
6. Doronina, S. O. et al. Development of potent monoclonal antibody auristatin conjugates for cancer therapy. Nat. Biotechnol. 21, 778-784 (2003).
7. Muldoon, L. L. & Neuwelt, E. A. BR96-DOX immunoconjugate targeting of chemotherapy in brain tumor models. J. Neurooncol. 65, 49-62 (2003).
8. Brown, W. L. et al. RNA bacteriophage capsid-mediated drug delivery and epitope presentation. Intervirology 45, 371-380 (2002).
9. Abbing, A. et al. Efficient intracellular delivery of a protein and a low molecular weight substance via recombinant polyomavirus-like particles. J. Biol. Chem. 279, 27410-27421 (2004).
10. Pattenden, L. K., Middelberg, A. P., Niebert, M. & Lipin, D. I. Towards the preparative and large-scale precision manufacture of virus-like particles. Trends Biotechnol. 23, 523-529 (2005).
11. Janes, K. A., Calvo, P. & Alonso, M. J. Polysaccharide colloidal particles as delivery systems for macromolecules. Adv. Drug Deliv. Rev. 47, 83-97 (2001).
12. Kotov, N. A. et al. Inverted colloidal crystals as three-dimensional cell scaffolds. Langmuir 20, 7887-7892 (2004).
13. Kukowska-Latallo, J. F. et al. Nanoparticle targeting of anticancer drug improves therapeutic response in animal model of human epithelial cancer. Cancer Res. 65, 5317-5324 (2005).
14. Singh, P., Gonzalez, M. J. & Manchester, M. Viruses and their uses in nanotechnology. Drug Dev. Res. 67, 23-41 (2006).
15. Hooker, J. M., Kovacs, E. W. & Francis, M. B. Interior surface modification of bacteriophage MS2. J. Am. Chem. Soc. 126, 3718-3719 (2004).
16. Wang, Q., Kaltgrad, E., Lin, T., Johnson, T. E. & Finn, M. Natural supramolecular building blocks: wild-type cowpea mosaic virus. Chem. Biol. 9, 805-811 (2002).
17. Chatterji, A. et al. New addresses on an addressable virus nanoblock: uniquely reactive lys residues on cowpea mosaic virus. Chem. Biol. 11, 855-863 (2004).
18. Portney, N. G. et al. Organic and inorganic nanoparticle hybrids. Langmuir 21, 2098-2103 (2005).
19. Henning, P. et al. Tumor cell targeted gene delivery by adenovirus 5 vectors carrying knobless fibers with antibody-binding domains. Gene Ther. 12, 211-224 (2005).
20. Gleiter, S. & Lilie, H. Cell-type specific targeting and gene expression using a variant of polyoma VP1 virus-like particles. Biol. Chem. 384, 247-255 (2003).
21. Chua, C. C., Geiman, D. E., Keller, G. H. & Ladda, R. L. Induction of collagenase secretion in human fibroblast cultures by growth promoting factors. J. Biol. Chem. 260, 5213-5216 (1985).
22. Yang, N. S., Burkholder, J., Roberts, B., Martinell, B. & McCabe, D. In vivo and in vitro gene transfer to mammalian somatic cells by particle bombardment. Proc. Natl. Acad. Sci. USA. 87, 9568-9572 (1990).
23. Gross, J. & Nagai, Y. Specific degradation of the collagen molecule by tadpole collagenolytic enzyme. Proc. Natl. Acad. Sci. USA. 54, 1197-204 (1965).
24. Thornberry, N. A. & Lazebnik, Y. Caspases: enemies within. Science 28, 1312-6 (1998).
25. Bauer, L. A., Birenbaum, N. S., & Meyer G. J. Biological applications of high aspect ratio nanoparticles. J. Mater. Chem. 14 (4), 517-526 (2004).
26. Angelescu, A., et al. Porous silicon matrix for applications in biology. Rev. Adv. Mater. Sci. 5, 440-449 (2003).
27. Pistel, K. F., Bittner, B., Koll, H., Winter, G., & Kissel, T. Biodegradable recombinant human erythropoietin loaded microspheres prepared from linear and star-branched block copolymers: Influence of encapsulation technique and polymer composition on particle characteristics. J. Control. Release 59 (3), 309-325, 1999.
28. Voskerician, G., et al. Biocompatibility and biofouling of MEMS drug delivery devices. Biomaterials 24 (11), 1959-1967, 2003.
29. Zakhidov, A. A., et al. Carbon structures with three-dimensional periodicity at optical wavelengths. Science 30, 897-901 (1998).
30. Visse, et al., Matrix metalloproteinases and tissue inhibitors of metalloproteinases: structure, function, and biochemistry. Circ. Res. 92, 827-39 (2003).

What is claimed is:

1. An agent delivery system for controlled and targeted delivery of an agent to a target site, comprising:
an agent delivery platform, including
a base layer; and
a collagen-containing layer coating the base layer; and
at least one agent for delivering to a target site, wherein the at least one agent is disposed within the Agent Delivery Platform, wherein the at least one agent is released when the Agent Delivery Platform is exposed to collagen-degrading compounds.

2. The agent delivery system of claim 1, wherein the base layer is selected from a crystal lattice, and an inverse replica of a crystal lattice.

3. The agent delivery system of claim 2, wherein the base layer is a silica crystal lattice.

4. The agent delivery system of claim 1, wherein the collagen-containing layer includes a second material, where interlocking junctions are provided between the collagen and the second material.

5. The agent delivery system of claim 4, wherein the second material is selected from: actin, vimentin, fibronectin, laminin, and proteoglycan.

6. The agent delivery system of claim 1, and further comprising a outer layer coating the collagen-containing layer.

7. The agent delivery system of claim 6, wherein the outer layer is a second collagen-containing layer.

8. The agent delivery system of claim 7, wherein the at least one agent is disposed in the outer collagen-containing layer.

9. The agent delivery system of claim 8, wherein at least one additional agent is provided and disposed within the agent delivery platform.

10. The agent delivery system of claim 9, wherein the at least one additional agent is disposed within the first collagen-containing layer.

11. The agent delivery system of claim 1, wherein the at least one agent is selected from: small molecules, proteins, peptides, nucleotides, gene-based agents, siRNAs, miRNAs, c-miRNAs, antagomers, genes, viruses, viral constructs, antibodies, chemoattractants, and cytokines.

12. An agent delivery system for controlled and targeted delivery of an agent to a target site, comprising:
an agent delivery platform, including
a base layer, and
a collagen-containing layer coating the base layer;
at least one agent for delivering to a target site, wherein the at least one agent is disposed within the agent delivery platform, wherein the collagen-containing layer degrades as a function of time when placed adjacent cells of the target site, thereby releasing the at least one agent to the target site.

13. The agent delivery system of claim 12, wherein the target site is in an individual.

14. The agent delivery system of claim 13, wherein the target site is in a mammal.

15. The agent delivery system of claim 13, wherein the target site is a wound.

16. The agent delivery system of claim 13, wherein the target site is cancerous.

17. The agent delivery system of claim 12, wherein the base layer is selected from a crystal lattice, and an inverse replica of a crystal lattice.

18. The agent delivery system of claim 17, wherein the base layer is a silica crystal lattice.

19. The agent delivery system of claim 12, and further comprising a outer layer coating the collagen-containing layer.

20. The agent delivery system of claim 19, wherein the outer layer is a second collagen-containing layer.

21. The agent delivery system of claim 20, wherein the at least one agent is disposed in the outer collagen-containing layer.

22. The agent delivery system of claim 21, wherein at least one additional agent is provided and disposed within the first collagen-containing layer.

23. The agent delivery system of claim 12, wherein the at least one agent is selected from: small molecules, proteins, peptides, nucleotides, gene-based agents, siRNAs, miRNAs, c-miRNAs, antagomeres, genes, viruses, viral constructs, antibodies, chemoattractants, and cytokines.

24. A method of delivering an agent to a target site of an individual in a controlled and targeted manner, comprising:
providing an agent delivery system according to claim 1 or claim 12;
placing the agent delivery system adjacent the target site of the individual; and
maintaining the placement of the agent delivery system for a period of time, such that the at least one agent is released to the target site.

25. The method of claim 24, wherein the at least one agent is selected from: small molecules, proteins, peptides, nucleotides, gene-based agents, siRNAs, miRNAs, c-miRNAs, antagomeres, genes, viruses, viral constructs, antibodies, chemoattractants, and cytokines.
